(19) United States
(12) Patent Application Publication
(10) Pub. No.: US 2015/0050621 A1
(43) Pub. Date: Feb. 19, 2015

(54) COLORED RESTORATIVE DENTAL FILLING SYSTEM

(71) Applicant: Kristy D. Formby, Newnan, GA (US)

(72) Inventor: Kristy D. Formby, Newnan, GA (US)

(21) Appl. No.: 14/178,300

(22) Filed: Feb. 12, 2014

Related U.S. Application Data
(60) Provisional application No. 61/865,475, filed on Aug. 13, 2013.

(51) Int. Cl. A61K 6/08; A61C 13/15

(52) U.S. Cl. CPC A61K 6/08; A61C 19/003
USPC 433/226

(57) ABSTRACT

A composition and method for use in restorative and cosmetic dentistry which transforms the appearance of treated teeth with permanent novelty colors.

This application claims the benefit of priority from U.S. Provisional Patent Application Ser. No. 61/865,475, filed Aug. 13, 2013.

### TECHNICAL FIELD OF THE INVENTION

The invention is a colored, restorative dental filling system that has utility in the field of cosmetic dentistry. The preparations and method of the invention are suitable for use in treatment of dental caries or cavities, for example in pediatric dentistry, where the system is used to create a permanent coloration of the dental patient's teeth in a novelty color of choice. The system is useful to reduce anxiety in the patients and to create a novel cosmetic effect that will positively affect the patient's attitude toward dental treatment.

The fear of dentistry or receiving dental care, called dentaphobia or odontophobia, is well recognized among adults and children. A significant proportion of children are known to exhibit dental fear, the causes of which may vary. According to a published article in the British Dental Journal, one possible cause of dental fear is lack of control. British Dental Journal, 187, 408-412 (1999). Each person, regardless of age, has an innate sense of boundary within which they expect and are socialized to exhibit personal control. The invasive nature of dental treatment may trigger a sense of loss of control, which in turn can lead to dental fear, and eventually to dentaphobia. Means for addressing the feeling of lost control include providing information, including with demonstration; offering control over the uncomfortable or unwanted stimulus; and by offering decisional control. The present invention seeks to address the problem of dental fear or dentaphobia in pediatric patients by use of a dental restorative system that has a novel, cosmetic effect and offers the patient decisional control as to the final appearance of the restoration.

The present invention further provides a means of novelty cosmetic enhancement that may be entertaining and serve as a fashion choice for the wearer.

### BACKGROUND ART

The field of cosmetic and restorative dentistry has included the use of various filler materials including metal amalgams, solid composite fillings and in the last two decades, flowable resin filling materials. Most, if not all, the materials used to create fillings for dental caries (cavities) that match or approximate the color of the teeth are selected from composite materials of polymeric composition. The goal of restorative dentistry in using composites, and the goal of using resinous materials in cosmetic dentistry, has always been to provide a filling or overall appearance of the tooth that matches the coloration, brightness and opacity of the existing dentition as much as possible. It would, in this art, therefore be an undesirable result for a resinous filling to stand out in appearance from the rest of the teeth when the teeth of the patient are viewed under natural lighting and in normal, everyday environments.

An article published by the Journal of the American Dental Association discusses the commonly used forms of composites, including packable and flowable resins, and describes the typical composition of flowable composite resins as including a polymer and filler. It describes most commercially available composites as being polyacrylate resins. The article goes on to state that composite-based resins should have a range of translucency and opacity that reflects that of enamel and dentin. Fortin, D., The Spectrum of Composites: New Techniques and Materials, J. American Dental Assn., Vol. 131, June 2000; page 288. Flowable resins are used as preventive resin restorations to fill small areas of caries in pits and fissures. Alternatively, a flowable can be used on top of a more robust filling, such as a conventional or composite packable filling. After the restoration, the entire occlusal surface is typically sealed with a sealant.

U.S. Pat. No. 4,150,485 discloses a flowable acrylate resin that is used to coat stains or hypoplasia on the teeth, to restore the natural color and fluorescence of the teeth and to restore worn or discolored composite restorations. The patent states the restorations so produced are virtually indistinguishable from natural teeth, and that the compositions of the invention have an enhanced ability to match the natural shading of the teeth. U.S. Pat. No. 5,094,619 describes a method of treating dental restorations, such as fillings, bridges, crowns and dentures with sucrose which caramelizes when heat is applied. The heat caramelization of the sucrose is completed just to the point where the filling will match closely the color of the adjacent teeth. U.S. Pat. No. 5,089,306 discloses a preformed, shrinkable tube device that can be put over a crown or dental construct, and which is formed of a glazing material formed of vitreous (glass) particles mixed in a carrier with a shading porcelain powder. The tube is put over the crown or dental construct and heat treated to shrink it over the construct; this process may be repeated, according to the patent, by repeating the process if the dental construct is too light (i.e. does not match closely to the adjacent teeth) after one application.

Generally, the process of restorative filling for dental caries involves identifying and defining the extent of the cavity, abrading the surfaces of the cavity and then inserting one or more appropriate filling materials. The filling materials are set by adhesive, or cured if curing is necessary to solidify and set them using an appropriate curing source, then the treated surface is polished. The polished surface or the entire tooth may then be sealed with a sealant to protect the integrity of the filling and the tooth itself. It is known in the art that many composite fillings match so closely the coloration of the tooth after this process is completed that the original location or extent of the filling cannot be detected by visual inspection, because the radiopacity of the filling is close to that of the natural teeth enamel and dentin. To address this issue, radiopacity agents are added to the filling materials during manufacture so that the extent and location of the fillings can be determined at a later date when the teeth are X-rayed. Common radiopacity agents are zirconium dioxide, barium oxide or ytterbium oxide.

It has not been known in the field of restorative or cosmetic dentistry to create a filling system that creates a permanent novelty coloration of the tooth or teeth being treated for the purpose of distinguishing the appearance of the restored tooth, for example using colors such as pink, blue or purple specifically for the purpose of causing the treated tooth to stand out in color from the color of the adjacent teeth. Indeed, the references mentioned above do not describe a composition or process that produces distinguishable, non-white, permanent novelty coloration to the tooth. Nor does the art contemplate using a cosmetic application during the necessary process of treating dental cavities as a means of reducing anxiety, dental fear or dentaphobia in pediatric dental patients. The present invention enables a novelty application which has desirable applicability and utility in the treatment of dental caries in primary teeth, by creating a specific, desired cosmetic appearance that is distinct from the surrounding teeth. The invention further provides a solution to the problem of anxiety and stress in child patients during dental treatment by providing a means of distraction, excitement and a sense of control over the outcome of the dental visit, thus creating a calming effect. The invention is further suitable for any patients desiring novelty coloration of the teeth.

### BRIEF DESCRIPTION OF THE DRAWINGS

FIG. 1 is a schematic representation of an exemplary process according to one embodiment of the invention for creating novelty colored dental restorations for primary teeth.

### BRIEF SUMMARY OF THE INVENTION

The invention provides novel colored composite fillings and methods for their use, particularly in the field of pediatric dentistry. Methods and materials for composite fillings of primary teeth have been formulated to provide a whiteness that matches the natural color of the teeth. There is an unsolved problem in the art that a choice of colors is not available the patient from which a non-white color can be selected at the patient's option. However, being able to choose a novelty color for the final appearance of the finished dentistry is exciting for a child, and being able to choose colors also helps to relieve the patient's anxiety about the planned dental work. The invention provides a choice of novelty colors that permanently color the filling and also the sealant so that the appearance of the tooth is changed to the desired novelty color, for example purple, pink, blue or red.

The invention therefore comprises a dental filling system for permanent, novelty coloration of teeth that includes one or more layers of a flowable material comprising a non-white, novelty colorant applied to the cavity of a tooth; one or more layers of a sealant comprising a non-white, novelty colorant having the same color as the colorant used in the flowable material also applied to the cavity, preferably over the one or more layers of flowable material; and optionally, one or more layers of a packable filling material beneath the flowable material and the sealant which includes a non-white, novelty colorant having the same color as the colorant used in the flowable material or in the sealant. Usually, the same color is used in the flowable composite, the sealant and the packable filling material.

The invention further comprises a method for filling a dental cavity that provides a permanent, non-white novelty coloration of the tooth. This method includes depositing at least one layer of flowable material comprising a non-white, novelty colorant over the filling material; curing the flowable material and filling; and depositing at least one layer of a sealant comprising a non-white, novelty colorant over the flowable material to provide a visible, permanent, non-white novelty cosmetic coloration of the filing and the tooth.

When the system is applied to the teeth, it provides a permanent, non-white, novelty coloration of the tooth which is readily visible. The coloration is permanent, therefore the system is suited for treatment of primary teeth in children as those teeth will naturally fall out over time and be replaced by permanent teeth. However, the invention may also be used by any patients seeking to obtain permanent novelty coloration of their teeth during the cavity filling process.

### DETAILED DESCRIPTION

The invention provides a range of colors of filling materials which are different from the traditionally selected white or off-white matching tones of composite materials used to approximate the color of the patient's natural dentition. Rather, the system and process of the invention may be manipulated, for example at the selection of the patient, to create a novelty appearance. Prior to the invention, children did not have a choice of colors that could be applied to their primary teeth when having fillings done. According to the invention, pediatric patients are given the choice to have a filling that may for example be blue, green or pink in color. The color may be selected from a range of standard colors, such as blue, red, pink, yellow, orange, green or purple, or tones thereof. The coloration may be adjusted to the patient's liking, for example to create pastel or deeper tones. Before the invention, such options were not available.

The invention is in one embodiment desirably used to treat primary teeth. Because the tooth is a primary tooth, as the child grows it will be lost as the permanent tooth begins to grow in. Therefore, the appearance of the permanent tooth will not be affected by the permanent novelty coloration applied to the primary tooth. However, it should be noted that this option for coloration is also available for permanent teeth as well at the patient's choice and selection for any desired reason.

The opportunity to have colored fillings and coatings applied to a primary tooth provides a positive and enjoyable experience for children who may otherwise experience anxiety during a visit to the dentist. The system and method also provides more choices for the dentist or oral surgeon in deciding how to restore the tooth. The patient can engage in the process by deciding on colors, and with parental consent as may be necessary, the treatment can be completed.

The filling materials may be selected from composite or polymeric, non-metallic materials conventionally available or supplied to dental professionals. According to an article published by the Journal of the American Dental Association, by definition a composite based resin material (hereinafter referred to as a composite) is a material that consists of two or more components. Composite resin manufacturers have not readily disclosed the exact composition of their formulations, however typical composites are composed of a resin or matrix, fillers, and interfacial phase to couple the filler with the matrix, and initiators for polymerization. The matrix phase may be composed of organic difunctional monomers. In conventionally used filling materials, the fillers are selected for hardness but also their ability to be finely polished, and may include aluminum, lithium or fumed silica to contribute hardness. Traditional resin fillings are made of composites that are available in a solid form for insertion into the cavity. Flowable composites have less viscosity than traditional resins and can flow into small crevices and tight undercuts of the cavity to provide a more completely occlusive fill while adapting virtually seamlessly to the contours of the tooth. A packable composite has higher viscosity, which allows it to be packed or forced into the small openings of a cavity without flow or migration to other parts of the tooth.

The term, “non-white, novelty colorant,” as used herein refers to pigments and colorants that are not white or white-based and not intended to produce colorations that match or approximate the white or off-white shades of natural teeth. Rather, the pigments and colorants suitable for use in the invention are chosen for their biologically atypical colorations in the finished product, such as primary colors or pastel tones such as pink, purple and blue. These colors are not naturally known in the dentition of humans, and are intended to create a deliberate contrast to the coloration of the natural teeth.

The non-white novelty colorants used in this invention may be selected from among any non-toxic, food grade, organic or synthetic dyes or pigments. The colorant may be added in liquid form to the various dental filling materials, and it may also be added in solid form if its particulate size is small enough to allow it to be finely and evenly dispersed to create the desired color. Examples of suitable coloring agents include liquid and solid food colors and FD&C colors in conventional shades and combinations; and food-grade dyes such as anthocyanins, astaxanthins, riboflavins, carotenoids, hydroxyanthraquinoids, chlorophylls and other naturally derived colorants; and combinations thereof. For example, food colors comprised of approved dyes, such as FD&C colors in a solution of propylene glycol or other food-grade base may be used. Exemplary FD&C dye colors approved for food use include FD&C Yellow #5, Blue #1 and Red #40, which may each be added to the substrate materials in varying proportions to produce deeper or lighter tones, and further combined together to produce secondary colors of varying intensity. The desired amount of colorant may range from a small amount on the tip of a toothpick, i.e. a fraction of a drop or a microgram amount, if solid, up to the amount required to achieve the desired coloration of the material. A different colorant may be applied in the different layers of the system according to the invention to create a final overall coloration or color effect such as gradations or shading. The colorant should not be added at an amount that would cause instability of the filling material, either by changing its viscosity, by causing it to separate, or by causing curing failures. The colors may be mixed to provide varied shades and tones from bright primary colors to pastel shades.

As used herein, the term “flowable material” refers to a flowable, composite resin material or a suitable substitute. The flowable material used in this invention may be selected from resin-based flowable composite materials. A non-limiting example of a suitable flowable composite is 3M ESPE Filter flowable restorative in shade A-1 (white to match the coloration of natural teeth), which is mixed with food color. The solid, non-flowable filling material of this invention may be comprised of traditional filling materials or of packable composites, which may be selected from resin based composites. An example of a packable composite that may be used in the invention is Prime-Dent light curable micro-hybrid composite in shade A-1, manufactured by Prime Dental Manufacturing, Inc., which is mixed with food color. A non-limiting example of a suitable sealant according to the invention is Natural Elegance sealant material, manufactured by Henry Schein, Inc., which is mixed with food color. Other resin-based sealants may be used.

In some patients, the flowable material may be the primary underlying filling material, and in such event it is inserted as the first filling layer in the cavity, optionally after a layer or adhesive has been applied. In deeper cavities, for example where there is significant breadth of the cavity or pulp exposure, a non-flowable filling material may be applied before use of the flowable material. In this respect, any suitable traditional polymeric white fillings or contemporary resin composites may be used. The non-flowable filling is typically supplied as a tapered stick, which at the distal end is finely pointed to allow its insertion into small diameters, and which progressively thickens in diameter along its length. Larger openings accommodate the thicker diameters. This filling material is inserted into the cavity, with or without a layer of adhesive as appropriate, and tamped down to ensure it has securely contacted the crevices before application of the flowable material.

The compositions of flowable composite, solid filling material and sealant may each be pre-mixed and sold commercially in the desired novelty colors, or they may be blended chair-side to produce the color and shade preferred by the dentist or patient. The amount of colorant determines the final color intensity of the composition. Primary colors of colorants mix well, and the amounts may be varied to produce pastel colors. Pastel colors may be more desirable for some patients. To achieve these colorations, a lesser amount of the colorant may be used, or a standardized amount of colorant in a premixed pastel shade may be incorporated into the blend.

In the methods according to embodiments of the invention, the surfaces of the cavity and the tooth are prepared according to accepted methods and standards of dentistry, for example by abrading and smoothing the surfaces, cleaning the cavity and acid-washing or etching the surfaces to be treated. As an optional step, a layer of adhesive may be applied before the filling materials are applied. Because resin-based materials are composed of monomers and an activator in a matrix, the material must be cured, usually by exposure to a light source of desired frequency, to activate polymerization of the resin and harden the material. The filling of the cavity itself may be accomplished solely by use of a flowable composite resin material, or it a solid filler or packable composite may be applied in the cavity before one or more layers of the flowable composite is applied. Each layer of material may require curing upon application. After these filling steps are completed, the sealant is applied. If a resin-based sealant is applied, it may also be subjected to a curing step.

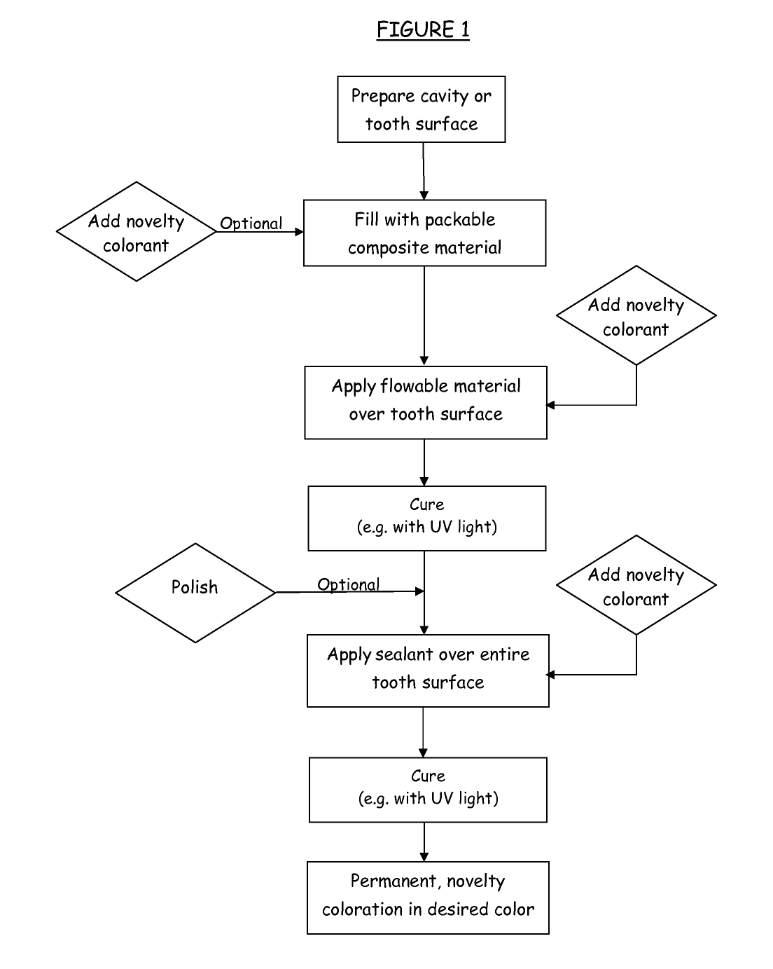

FIG. 1 demonstrates certain steps in the process of applying the system according to another embodiment of the invention. As shown in the schematic, the tooth is prepared and if necessary, a packable composite or other filler material is inserted into the cavity if it is deep or if there is significant risk of or actual root exposure. Depending on the type of packable filler material, it can optionally be colored with the same food coloring (or one that is compatible) that is used in the coloring of the flowable material and optionally the sealant. The filler material is cured, if curing is required. The flowable material is mixed with the colorant before being applied and then cured. The surface so formed may be sanded, shaped or polished, as needed, before application of the sealant. The sealant is mixed with the same colorant, applied and then cured to finish the application. It should be understood that the colorant may be pre-mixed with any or all of these materials at the point of manufacture or at the point of use.

### EXAMPLE 1

A pea-sized amount, approximately 0.5 to 1.0 grams of a light curable microhybrid packable composite material is blended with about 0.1 to 0.2 drops of liquid food coloring, about 0.005 to 0.010 ml, using a spatula and mixing pad. The resulting pastel-colored blend can be used to pack a cleaned and prepared dental cavity to which a layer of a suitable adhesive has been applied. The filling is cured using a suitable amount of exposure to a curing light. Approximately 0.5 to 1.0 grams of flowable composite is blended with 0.1 to 0.2 drops of food coloring in the same shade as used for the packable composite, and applied with a syringe or applicator gun over the surface of the filling and the surrounding surface of the tooth. Each applied layer(s) of flowable composite is cured. Approximately 0.5 to 1.0 ml of a sealant is mixed with 0.1 to 0.2 drops of food coloring in the same shade and applied to the entire surface of the tooth, then cured. The completed result provides a permanent pastel shading to the tooth.

Compositions according to the method and formula proportions of the invention have been formulated with vegetable dyes; however it was observed that the more diluted pigmentation and color intensity of such dyes required more volume of dye to be added to the flowable material and sealant. Larger volumes of added fluids may compromise the stability of the base material, and therefore in this respect vegetable dyes in standard concentration are not as preferable as synthetic food colorings suitable for food use.

It is to be understood that the amounts of the materials and colorant may be varied according to what is needed for that particular dental treatment and the desired coloration. Generally, for curing purposes a quartz halogen, LED light with LED curing light with a light energy emission of 600 mW/cm², or a plasma arc (PAC) curing light with a curing time of 5 seconds may be used.

### INDUSTRIAL APPLICABILITY

The invention is applicable in the fields of restorative and cosmetic dentistry. It is particularly useful in pediatric dentistry as a way to engage children in the process of dental treatment and dispel patient anxiety.

The foregoing description of specific embodiments and examples is not intended to be limiting on the scope of this disclosure, but rather to be illustrative of the broad concepts embodied by this invention. Those skilled in the art will appreciate that the present invention contemplates the various embodiments of the invention herein described as well as equivalents thereof. However, those skilled in the art will also appreciate that the scope of this invention should be measured by the attached claims as well as by the specific embodiments identified.

1. A dental filling system for permanent, novelty coloration of teeth comprising
a. One or more layers of a coating of a flowable material comprising a non-white, novelty colorant;
b. One or more layers of a sealant comprising a non-white, novelty colorant having the same color as the colorant used in the flowable material; and
c. Optionally, at least one layer of a packable filling material which includes a non-white, novelty colorant having the same color as the colorant used in the flowable composite material or in the sealant;
wherein the colorant in the one or more layers provides a permanent, non-white, novelty coloration of the tooth to which the system is applied.

2. The system of claim 1 wherein the colorants are selected from the group consisting of food grade pigments, food colors, and combinations thereof.

3. The system of claim 1 wherein the colorant is a food color.

4. The system of claim 1 wherein the flowable material is selected from the group consisting of resin-based, flowable composites.

5. The system of claim 1 wherein the sealant is selected from the group consisting of resin-based sealants.

6. The system of claim 1 wherein the packable filling material is selected from the group consisting of high-viscosity, packable resin-based composites, conventional composites, and combinations thereof.

7. The system of claim 1 further comprising an adhesive.

8. The system of claim 1 wherein a different non-white novelty colorant is used in each of the flowable material, sealant and packable filling material.

9. A method of filling a dental cavity that provides a permanent, non-white novelty coloration of the tooth comprising:
a. Optionally depositing and then curing at least one layer of a packable filling material into the cavity;
b. Depositing at least one layer of flowable material, which comprises a non-white, novelty colorant, over the filling material;
c. Curing the flowable material;
d. Depositing at least one layer of a sealant comprising a non-white, novelty colorant over the flowable material; and
e. Curing the sealant;
to provide a dental coating comprising a visible, permanent, non-white novelty cosmetic coloration of the filing and the tooth.

10. The method of claim 9 wherein the novelty colorant is mixed into any of the packable filling material, flowable composite material or sealant to provide the desired intensity of color immediately before it is applied to the tooth.

11. The method of claim 9 wherein the tooth is a primary tooth.

12. The method of claim 9 wherein the non-white novelty colorants are food grade pigments or food colors selected from the group consisting of primary, secondary or tertiary colors.

13. The method of claim 9 further comprising applying a layer of adhesive to the surface of the tooth before applying the flowable composite material or the packable filling material.

14. A dental coating formed by the process according to claim 9.

15. A dental coating formed by the process according to claim 13.
